(19) United States
(12) Patent Application Publication
(10) Pub. No.: US 2026/0041350 A1
(43) Pub. Date: Feb. 12, 2026

(54) OPTICALLY PUMPED MAGNETOMETRY TREADMILL

(71) Applicant: Father Flanagan's Boys' Home Doing Business as Boys Town National Research Hospital, Omaha, NE (US)

(72) Inventors: Max J. Kurz, Boystown, NE (US); Uyen Nguyen, Boystown, NE (US); Alexa Wildy, Boystown, NE (US)

(21) Appl. No.: 19/292,386

(22) Filed: Aug. 6, 2025

Related U.S. Application Data
(60) Provisional application No. 63/679,735, filed on Aug. 6, 2024.

(51) Int. Cl. A61B 5/246; A61B 5/00; A63B 22/02; A63B 69/00

(52) U.S. Cl. CPC A61B 5/246; A61B 5/6803; A63B 22/0285; A63B 69/0064; A61B 2562/0223

(57) ABSTRACT

A walking apparatus, e.g., a treadmill, which can be used in an optically pumped magnetometry (OPM) environment to measure the cortical dynamics of toddlers and adults while walking includes unique features. The treadmill is free of moving components and also free of magnetic metal. The treadmill includes a sloped portion that, in conjunction with a pair of designed shoes, has a low coefficient of friction to essentially allow the shoes to slide along the surface of the treadmill, which mimics a normal gait. The treadmill and shoes allow a user to be measured using an OPM to collect brain data during the gait.

### CROSS REFERENCE TO RELATED APPLICATIONS

This application claims priority under 35 U.S.C. § 119(e) to provisional patent application U.S. Ser. No. 63/679,735, filed Aug. 6, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to the field of neuroscience. More particularly, but not exclusively, the present disclosure relates to equipment that can be used in conjunction with neuroimaging machines, such as optically pumped magnetometers.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

OPM-MEG (Optically Pumped Magnetometers-Magnetoencephalography (MEG)) is a new non-invasive technology for imaging brain function in real-time with several advantages over traditional MEG.

Magnetoencephalography (usually abbreviated as MEG) is a non-invasive technology for imaging brain function in real time. The approach is based on the measurement of magnetic fields generated (mainly) by synchronous dendritic current flow through neuronal assemblies. Therefore, it is a direct measurement of brain activity. Mathematical modeling of these fields permits the development of three-dimensional images (termed source reconstruction) depicting the moment-to-moment variations in electrical activity as the brain responds to different experimental circumstances or cognitive demands.

It has been recognized that fine and gross motor skill development is an emerging process that matures by the age of 7-8 years. Yet, the neural underpinnings that govern the development of these motor skills remains an uncharted scientific frontier. A plethora of MEG experiments with adults have overwhelmingly verified that the production of gross and fine motor actions involves frequency specific changes in the strength of the sensorimotor cortical oscillations in the beta and gamma frequency range. The few investigations that have been conducted with children younger than 7-years of age have inferred that the respective sensorimotor cortical oscillations are weaker and may involve other compensational cortical areas when compared with adults. Other MEG studies have also inferred a similar developmental timeline exists for the somatosensory cortices by suggesting that the strength of the evoked somatosensory cortical activity matures when children are school-aged.

Altogether, these investigations suggest that the emergence of fine and gross motor skills are facilitated by fundamental changes in the cortical dynamics from 0 to 7 years of age. However, all of these prior investigations lack sufficient rigor to fully support these conclusions, as they were based on small sample sizes and the data was acquired with MEG machines designed to fit the adult head size. Essentially, the changes in the strength of the sensorimotor signals noted in the prior investigations conducted with children under 7-years of age might be partly due to the inability to the reliably measure the sensorimotor cortical oscillations with an adult sized machine since the strength of the measured neural signal falls off by the square of the distance the sensor is from the neural source. Furthermore, the prior data might not have adequate signal-to-noise because the conventional MEG methods necessitate a higher number of trials, which can be problematic when imaging less compliant toddlers and young children.

Recently, the listed inventors completed the largest MEG neuroimaging study to date (N=95) that evaluated the differences in the sensorimotor cortical dynamics in a cross-section of children and adolescents in the age range of 9-15 years. This experiment involved the youth performing a stimulus cued button press motor action with their right-hand. Similar to what is seen in adults, the beamformed images showed that the youth exhibited a characteristic beta (16-24 Hz) sensorimotor event-related desynchronization (i.e., power reduction) prior to and during movement in the contralateral motor hand knob region, as well as a 18-24 Hz post movement beta rebound (PMBR; power decrease) following the completion of the motor action (FIG. 1A). Furthermore, a succinct gamma (74-84 Hz) event related synchronization (ERS; power increase) occurred at movement onset (FIG. 1B). Importantly, our results showed that older youth tended to have a stronger PMBR (r=0.30, p=0.013) and a weaker gamma ERS (r=−0.26, p=0.027), implying that there are changes in the sensorimotor cortical dynamics across the formative years of adolescent development. It is expected that the changes in the respective cortical dynamics to be even more prominent across the 0-7 year age time frame.

The MEG preliminary experimental work also evaluated the strength of the evoked somatosensory cortical activity in a cross-section of children and adolescents that spanned the 11-to-19-year age range (N=22). The experimental paradigm involved electrical stimulation of the right tibial nerve at the motor threshold. As expected, the grand-averaged standardized low-resolution brain electromagnetic tomography (sLORETA) images revealed that the peak neural response emanated from the contralateral somatosensory cortices (FIG. 2). We subsequently extracted neural time course from the peak voxel and evaluated the possible relationship between a youth's age and the strength of the somatosensory response. The results suggest that the amplitude of the somatosensory cortical response tended to decrease with age (FIG. 2; r=−0.30; p=0.085), implying that there are potentially subtle changes in the refinement of the somatosensory cortical dynamics during adolescents. However, these preliminary results also imply that there are likely more prominent changes in these dynamics before 7-years of age.

Although the MEG preliminary studies are enlightening, they highlight that much of the insight on the neural sources surrounding the development of fine and gross motor skills in young children has relied on simple surrogate tasks (i.e., button press, sensory stimulation paradigms) due to the MEG and fMRI environment. Bluntly, the surrogate tasks might lack the necessary ecologic validity for adequately advancing the understanding of the neurophysiological mechanisms underlying the sensorimotor integration involved in the emergence of the motor milestones across development. Hence, this is one of the reasons the OPM technology is central to the overall theme of the present disclosure, as it can be used to quantify the neural dynamics during realistic motor tasks.

Evidently, there are substantial technical challenges with the design of ecologically valid neuroimaging paradigms that will robustly reflect the emerging changes in the cortical dynamics across development. In preparation for achieving this uncharted scientific vista, a series of experiments that were focused on the development of advanced neuroimaging methods that could directly measure sensorimotor cortical activity during gait and could be readily ported into the OPM experimental environment were brainstormed. Performing neuroimaging during gait obviously poses several challenges due to artifacts created by the large body movements and the collisions of the heel with the ground. Initially, a 64-channel portable EEG system was used to develop a neuroimaging pipeline that could be used to measure the somatosensory cortical activity induced by an electrical stimulation that was applied to tibial nerve of the participants (N=20) during the following experimental conditions: 1) sitting and 2) walking on a conventional treadmill. The experimental results showed that for the sitting condition the stimulation induced changes in the gamma (40-80 Hz; 40-100 ms) and theta (4-8 Hz; 50-300 ms) sensorimotor cortical oscillations (FIG. 3A). However, the gamma oscillations seen during the sitting condition was markedly absent or gated during gait, while the theta oscillations remained prominent (FIG. 3B). A follow-up study was conducted to provide further verification that the source reconstruction methods reliably quantify somatosensory cortical activity. For this follow-up investigation, the participants (N=26) underwent either stimulation of the median or tibial nerve under the following experimental conditions: 1) sitting quietly and 2) walking on a conventional treadmill (i.e., 4 conditions total). When the tibial nerve stimulation was applied to subjects while they were sitting (FIG. 4A) or walking on the treadmill (FIG. 4B), the theta activity was seen in the contralateral leg region of the somatomotor cortices. In addition, the theta activity resided in the motor hand knob region of the contralateral hemisphere when the median nerve was stimulated as participants sat quietly (FIG. 4C), and as they walked on the treadmill (FIG. 4D). These expected somatotopic changes in the anatomical locations clearly show that the source reconstruction pipeline reliably images the somatosensory cortical activity during an ecologically valid task such as gait. Transferring these pioneering methods into the OPM environment can vastly improve the source image spatial resolution and number of trials that are necessary to acquire valid data from children less than 7 years of age.

However, there are issues that exist with current equipment, such as treadmills and other components in use with an OPM. Belt motion, a motor, and magnetic metal parts that comprise traditional treadmills and gym equipment will create large artifacts that will contaminate the sensors of the OPM and render the data unusable.

Thus, there exists a need in the art for systems and/or apparatus that overcome the issues presented to allow better data to be acquired using an OPM with children in a moving manner.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to provide a walking apparatus that is safe to use in an OPM environment to obtain information during use of the OPM. For example, the walking apparatus will be free of magnetic metals and free of moving parts to avoid data contamination.

It is still yet a further object, feature, and/or advantage of the present disclosure to provide a walking apparatus that can be used with children in a consistent manner.

The walking apparatus/system disclosed herein can be used in a wide variety of applications. For example, while it can be used with children and adults in an OPM environment, it is envisioned that people of all ages. In addition, the apparatus/system can be used with other neuroimaging devices (i.e., fNIRS, EEG).

It is preferred that the apparatus be safe, cost effective, and durable. For example, the apparatus can be adapted to resist excessive heat, static buildup, corrosion, and/or mechanical failures (e.g., cracking, crumbling, shearing, creeping) due to excessive impacts and/or prolonged exposure to tensile and/or compressive forces acting on the apparatus.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the walking apparatus/system.

Methods can be practiced which facilitate use, manufacture, assembly, maintenance, and repair of a walking apparatus/system which accomplish some or all of the previously stated objectives.

According to some aspects of the present disclosure, a walking system for use with an optically pumped magnetometer comprises a walking surface comprising a center and upward sloping surfaces extending away from the center; wherein the walking surface comprises a low-friction material; and a frame adjacent to the walking surface, the frame including a harness for a user to aid in controlling the location of the user relative to the walking surface.

According to at least some aspects of some embodiments, the system further comprises shoes for lowering the coefficient of friction while contacting the walking surface.

According to at least some aspects of some embodiments, the coefficient of friction between the shoes and the walking surface is about 0.03-0.40.

According to at least some aspects of some embodiments, the harness is a waistband, chest, or full body harness.

According to at least some aspects of some embodiments, the walking surface is circular-shaped.

According to at least some aspects of some embodiments, the upward sloping surfaces of the walking surface is substantially the same slope around the center.

According to at least some aspects of some embodiments, the walking surface comprises a low-friction polymer.

According to at least some aspects of some embodiments, the system is substantially free of ferromagnetic materials.

According to still additional aspects of the disclosure, a method of collecting data using an optically pumped magnetometer while a user moves comprises connecting a user to the optically pumped magnetometer; having the user walk on a walking apparatus, wherein the walking apparatus comprises a walking surface comprising a center and upward sloping surfaces extending away from the center and the walking surface comprising a low friction material; and maintaining the user's position on the walking apparatus with a harness connected to a frame.

According to at least some aspects of some embodiments, the method further comprises providing shoes to the user, wherein the shoes comprise a polytetrafluorethylene outer sole for reducing the coefficient of friction in contact with the walking surface.

According to at least some aspects of some embodiments, the method further comprises collecting data via the optically pumped magnetometer as the user walks on the walking surface.

According to at least some aspects of some embodiments, the step of connecting the user comprises placing a helmet with sensors on the user's head.

According to at least some aspects of some embodiments, the walking apparatus is circular-shaped.

According to at least some aspects of some embodiments, the upward sloping surfaces of the walking surface is substantially the same slope around the center.

According to at least some aspects of the disclosure, the center of the walking surface has substantially zero slope.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A shows grand-averaged beamformed images for the post-movement beta rebound (PMBR), with the lower panel depicting scatter plots showing the relationship between age and the peak amplitude of the PMBR.

FIG. 1B shows grand-averaged beamformed images for the gamma event related synchronization (ERS), with the lower panel depicting scatter plots showing the relationship between age and the peak amplitude of the gamma ERS.

FIG. 2 is a graph depicting neural time-course of the evoked somatosensory cortical activity extracted from the peak voxel in the grand-average sLORETA image (shown in inset). A scatterplot depicting the negative relationship between the peak somatosensory cortical activity and age is also shown in the inset.

FIG. 3A shows grand averaged time-frequency spectrograms from an electrode near the sensorimotor leg region for the sitting.

FIG. 3B shows grand averaged time-frequency spectrograms from an electrode near the sensorimotor leg region for the treadmill walking.

FIG. 4A is a grand average beamformer image for the theta (4-8 Hz) activity seen when a foot was stimulated for the sitting condition.

FIG. 4B is a grand average beamformer image for the theta (4-8 Hz) activity seen when a foot was stimulated for the walking condition.

FIG. 4C is a grand average beamformer image for the theta (4-8 Hz) activity seen when a hand was stimulated for the sitting condition.

FIG. 4D is a grand average beamformer image for the theta (4-8 Hz) activity seen when a hand was stimulated for the walking condition.

FIG. 5 is a depiction of a toddler walking on a walking system according to aspects of the present disclosure and wearing an OPM helmet system to measure the cortical dynamics during gait.

FIG. 6A is a graph showing horizontal ankle motion displacement for walking on a traditional treadmill and a walking system for use with an OPM as disclosed herein.

FIG. 6B is a graph showing horizontal knee motion displacement for walking on a traditional treadmill and a walking system for use with an OPM as disclosed herein.

FIG. 6C is a graph showing vertical ankle motion displacement for walking on a traditional treadmill and a walking system for use with an OPM as disclosed herein.

FIG. 6D is a graph showing vertical knee motion displacement for walking on a traditional treadmill and a walking system for use with an OPM as disclosed herein.

FIG. 7A is a graph showing grand averaged time-frequency spectrograms while walking on a walking system as disclosed herein.

FIG. 7B is a beamforming image of the stimulation induced theta ERS was in the leg region of the somatosensory cortices from the gait in FIG. 7A.

FIG. 7C is a graph showing the neural time-course that was extracted from the peak voxel of the beamformed image of FIG. 7B showed that stimulation induced a robust somatosensory response.

FIG. 8 is a front view of a walking system for use with an OPM according to aspects of the present disclosure.

FIG. 9 is another view of a walking system for use with an OPM and showing a user connected to and walking on the system.

FIG. 10 is a cross sectional view of a walking surface of a walking system according to aspects of the present disclosure.

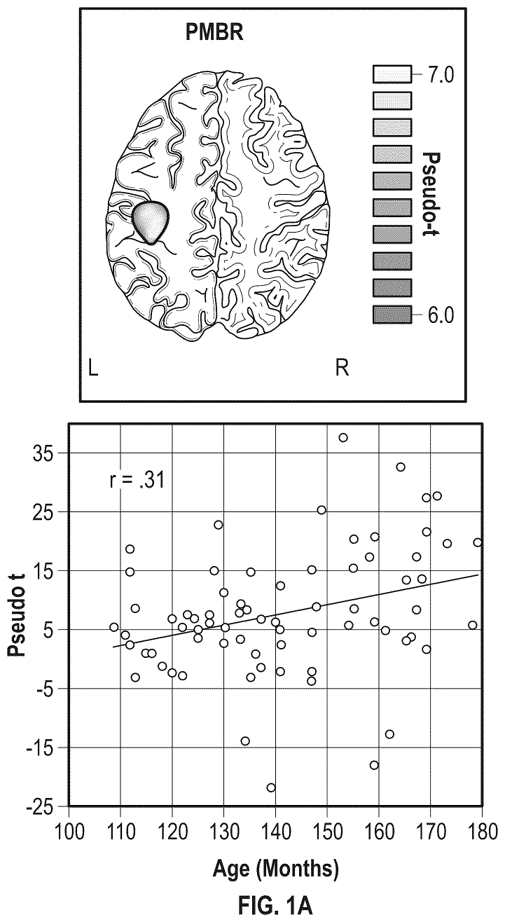

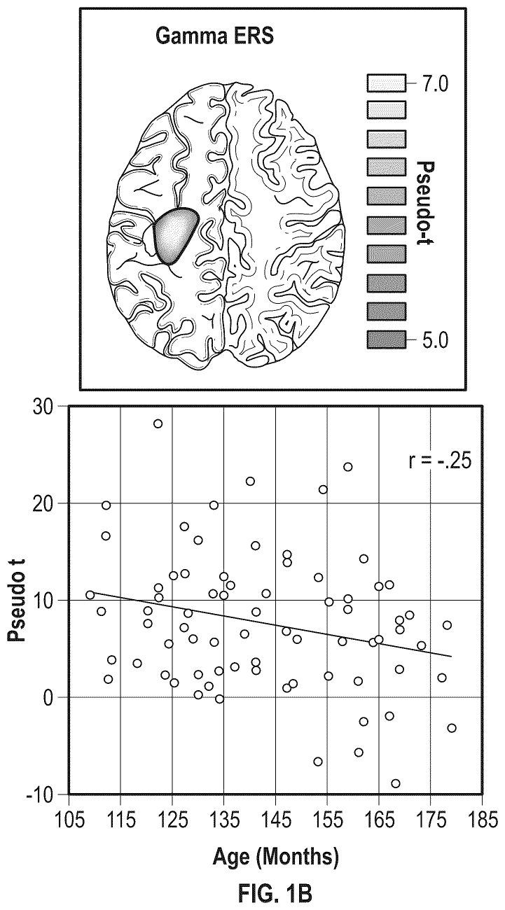

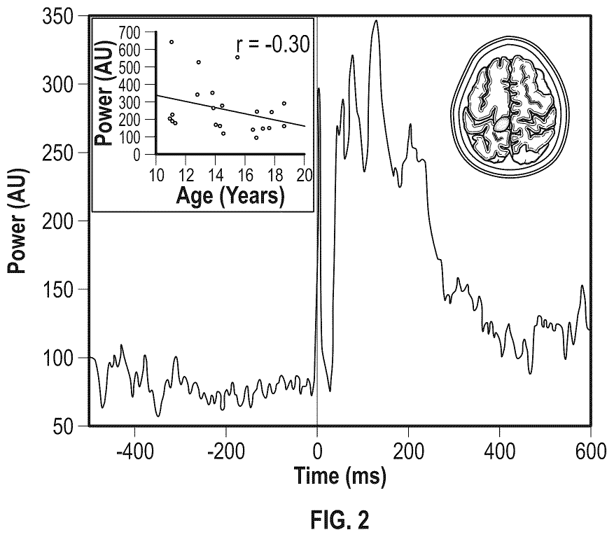

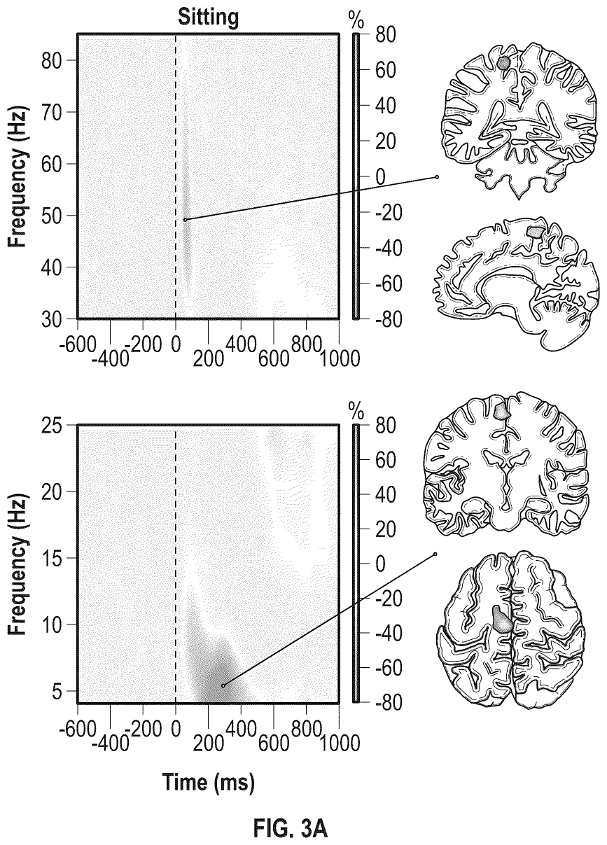

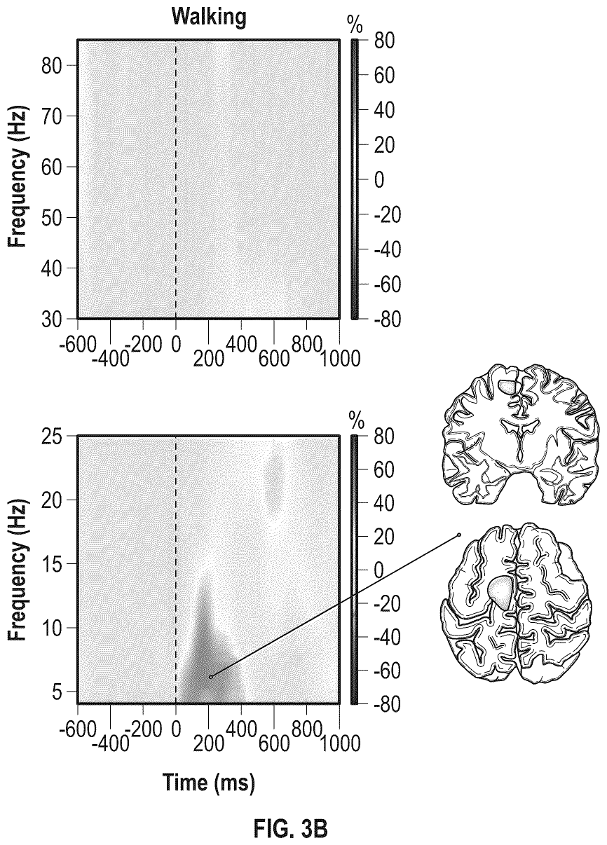

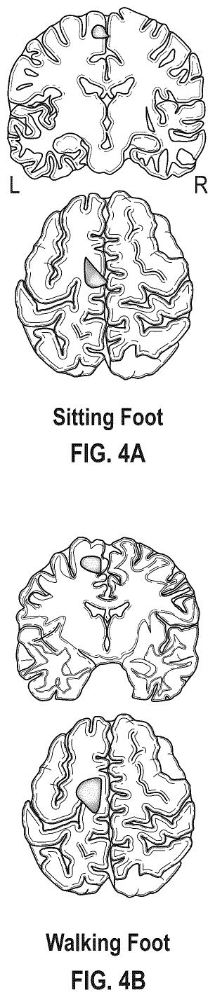

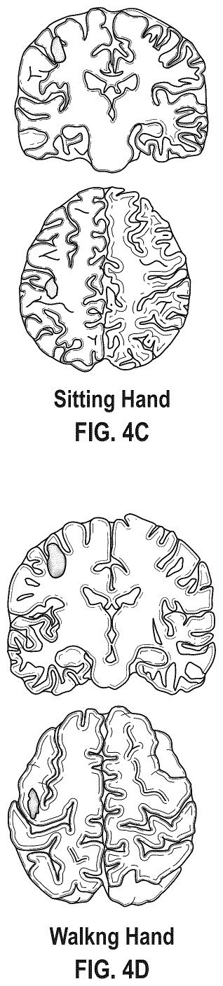

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The “center” of the apparatus for the present disclosure is defined as a part of the surface with substantially zero slope and/or the location on the walking surface where a user will stand when not in motion.

As has been understood, there is significant brain data that can be obtained from measuring movement, such as gait, for both children and adults while using an optically pumped magnetometer (OPM). To preserve space, treadmills or similar walking devices would be preferred as they allow for movement without a participant leaving a room in which the OPM or OPM device (e.g., helmet) is housed. However, there are issues with traditional treadmills and the like, as they include moving parts, motors, and a good deal of magnetic metal. These could all affect the data collection, rendering it basically useless.

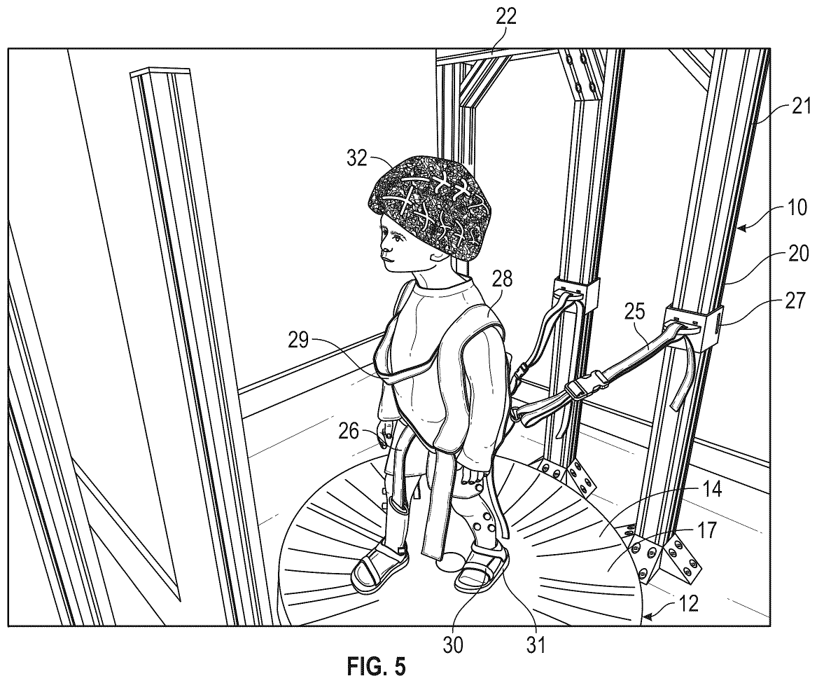

Therefore, according to at least some aspects and/or embodiments of the present disclosure, a walking system 10 is provided, which is shown in FIG. 5. As will be understood, the walking system 10 shown in the figures overcomes the technical challenges of traditional treadmills by engineering a walking system, e.g., a stationary treadmill, that can be used in the OPM environment for assessing the cortical dynamics of all users, including children younger than 7 years of age. The walking system 10 is a stationary device, which also can be referred to as a motorless treadmill deck that comprises a low-friction surface with a radius of curvature that creates a subtle elevation in the distal deck surface. The deck elevation profile causes the foot to slide backward towards the center of the deck upon heel-contact, which enables the participant to walk in-place.

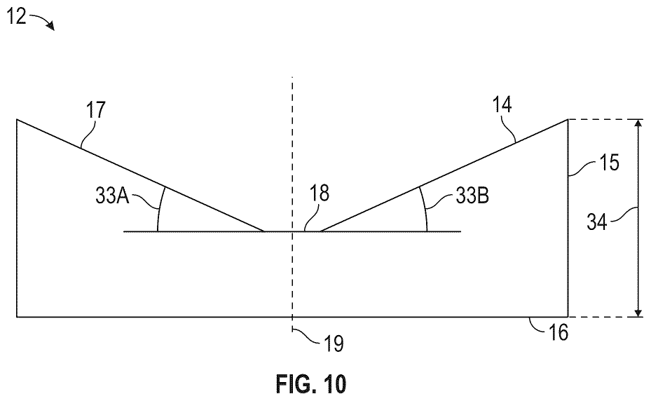

For example, as shown in FIG. 5, the walking system 10 (also referred to as an OPM treadmill or motorless treadmill deck), includes a walking surface 12. The walking surface or deck 12 includes a top portion 14, side 15, bottom 16 (see, e.g., FIG. 10), and an upward sloping surface 17 extending from a center portion 18. According to at least embodiments, the walking deck 12 is circularly shaped, wherein an axis 19 extends through the center 18 of the deck 12. However, it should be noted that the surface can be any geometrical shape that allows a sloped portion to extend from another portion that would allow a user to walk in place along the sloped portion to simulate walking. It should be appreciated that the slope can be linear or non-linear, and the present disclosure is intended to cover any and all generally positive slopes moving away and upward from a center or non-sloped portion of the walking deck.

As shown best in FIG. 10, the walking deck 12 includes an upward sloping surface 17 extending fully or partially around the center 18, such as in a particular direction. However, if the deck is non-circular (e.g., square or rectangularly shaped), there may be upward sloping surfaces in one or more directions. Additionally, as shown in FIG. 10, the upward sloping surface 17 extends generally from the center 18. According to at least some embodiments, the slope 33 is consistent circumferentially around the center 18 and/or axis 19. Additionally, the slope may be varied circumferentially or just extend in one or more directions. As shown in FIG. 10, this would be indicated by the first slope 33A being substantially equal to the second slope 33B, such that there is a common slope for the deck 12. However, it should also be considered that the upward slope vary or include different slopes, which may be useful for participants/users of different size and/or age.

For example, a first slope 33A may include a slope that has been configured for people having a stride length of height of certain threshold. The deck 12 may also include one or more additional sloped portions that are configured for people of different sizes or having different stride lengths. This would allow a single walking surface 12 to be used with people of varying heights, ages, abilities, etc. This would provide immense benefits to reduce the number of components needed in testing groups of people.

Further aspects of the walking surface 12 include a composition or layer of reduced friction material. This can be an overmolded portion or a layer that is added to the top. As will be understood, the layer is included to reduce the friction between a user (such as the sole of a user's shoe) and the sloped surface 17 to allow the shoe to essentially slide along the surface. The sole and reduced friction will reduce the impact of a shoe on the surface, which will also reduce any “noise” that could affect the data being acquired via the OPM during the movement of the user. The reduced friction surface can take many forms, including, but not limited to a low-friction plastic or other polymer.

To further reduce the friction between the walking surface 12 and the user 28, shoes 30 can be provided with manipulated soles 31. The soles 31 of the shoes 30 can comprise a reduced friction element to allow the soles 31 to better slide or glide on the surface 12, including the sloped portion. Shoes 30 with the manipulated soles 31 are shown in both FIGS. 5 and 9.

According to at least some aspects of the disclosure, the shoes 30 have a polytetrafluorethylene (PTFE) outer sole 31 to further reduce the coefficient of friction while the foot is in contact with the deck surface 12. According to some aspects, the material of the soles and walking deck can be any material(s) having a coefficient of friction between 0.03-0.40. One example of a suitable material is chemical-resistant slippery Teflon® PTFE, which has a coefficient of friction range of approximately 0.05-0.08. However, other materials capable of allowing shoes to slide or glide along the sloped surface 17 of the walking surface 12 are to be considered a part of the present disclosure, and the exact material is not limiting on the disclosure. This can include, but is not limited to additional lubricants, which may be liquid, semi-liquid, or solid.

Still additional portions of the walking system 10 of the present disclosure are shown in FIGS. 5, 8, and 9, and include a frame portion 20. The frame 20 is used to aid in positioning and maintaining a position of a user 28, and also to mitigate the user from falling down or otherwise becoming out of control. As noted, the shoes 30 and surface 12 are designed to be relatively slick and have a low coefficient of friction. This may make it difficult to maintain posture and/or positioning, especially when first using the system 10. Therefore, the frame 20 will aid in the positioning and safety of the system.

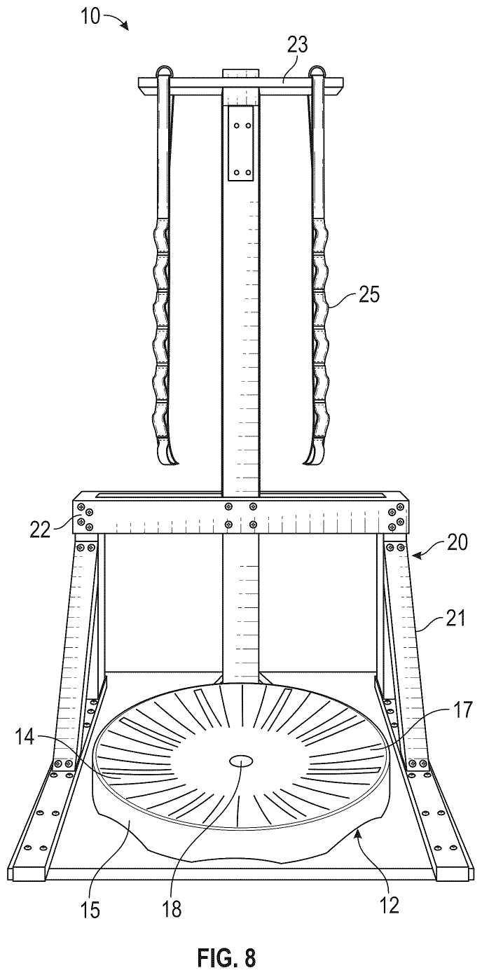

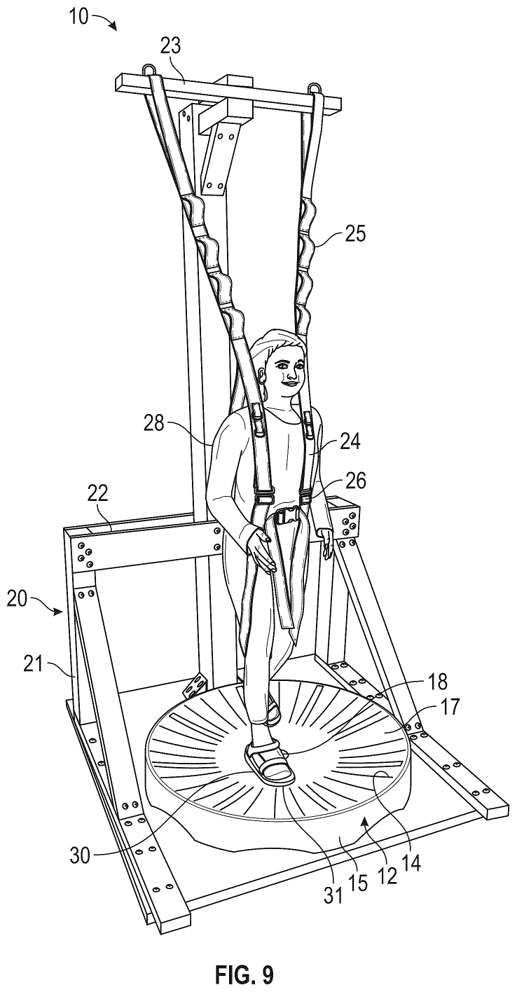

The frame 20 includes legs 21, which may be aluminum, wooden, or other non-ferromagnetic and non-paramagnetic materials. The legs 21 are designed to be either around the walking surface 12 (e.g., FIGS. 8-9) or otherwise adjacent to the surface 12 (e.g., FIG. 5). The legs 21 include vertical or slightly angled from vertical portions to raise the frame. One or more crossbars 22 may be included to connect the legs 21, and may include an upper crossbar 23, which will be described in more detail herein.

As shown in both configurations, a harness 24 can be connected to the frame 20 and the user 28. Referring to FIG. 5, a waistbelt harness 24 is connected to an aluminum frame 20 that is used to ensure the participant 28 remains on the deck 12 while walking on the OPM treadmill. The harness in the figure includes straps 25 connected to the legs 21 and a harness portion 24 including a waist belt 26 that is positioned on the participant user 28. The straps 25 are connected to the legs 21 via height adjustable connectors 27, which allow for the straps to be adjusted according to the height of the user participant 28. The connector can be any mechanical or non-mechanical fastener that allows for releasable connection for the straps to allow for the height adjustment.

In general, a mechanical fastener is a device that is used to mechanically join or fasten two or more objects together. In general, fasteners are used to create non-permanent joints or connections; that is, joints that can be removed or dismantled without damaging the joining components. General types of mechanical fasteners can include threaded (bolts, screws, nuts, threaded inserts, studs, etc.) or non-threaded (keys, pins, retaining rings, etc.). Additional fasteners can include, but are not limited to nails, rivets, and the like. Non-mechanical fasteners may include adhesives, fittings, clearance fittings, friction fittings, compression fittings, transition fittings, snaps, snap fits, hook and loops, joints, and the like. For simplistic purposes, screws, nuts, bolts, pins, rivets, staples, washers, grommets, latches (including pawls), ratchets, clamps, clasps, flanges, ties, adhesives, welds, any other known fastening mechanisms, or any combination thereof may be used to facilitate fastening, may be used for any of the connections described herein and all are to be considered swappable with one another for any of the attachment, connection, and/or fastening of components, either temporarily or permanently. It is further considered that any combination of any of the listed mechanical and/or non-mechanical fasteners or methods of fastening are to be considered a part of the disclosure.

Similarly, the system 10 shown in FIGS. 8 and 9 also include a harness 24. The harness shown in these figures includes straps 25 extending from an upper crossbar 23 and connected to a waist belt style harness 26 that is are positioned on the user participant 28 (see, e.g., FIG. 9). Again, this keeps the positioning of the user relative to the walking surface and also adds a layer of safety for use of the device.

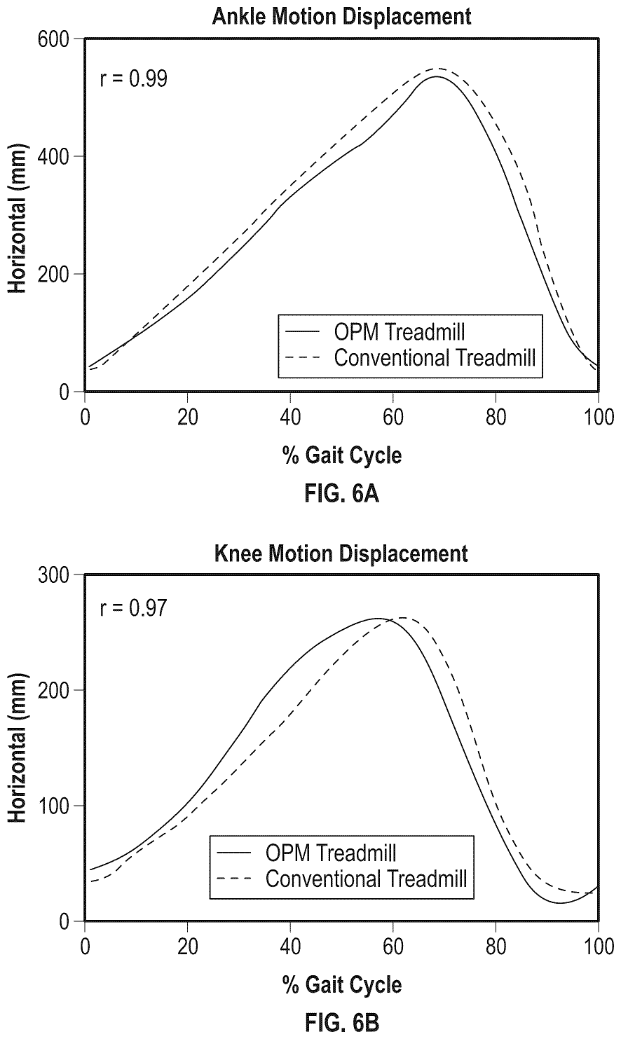

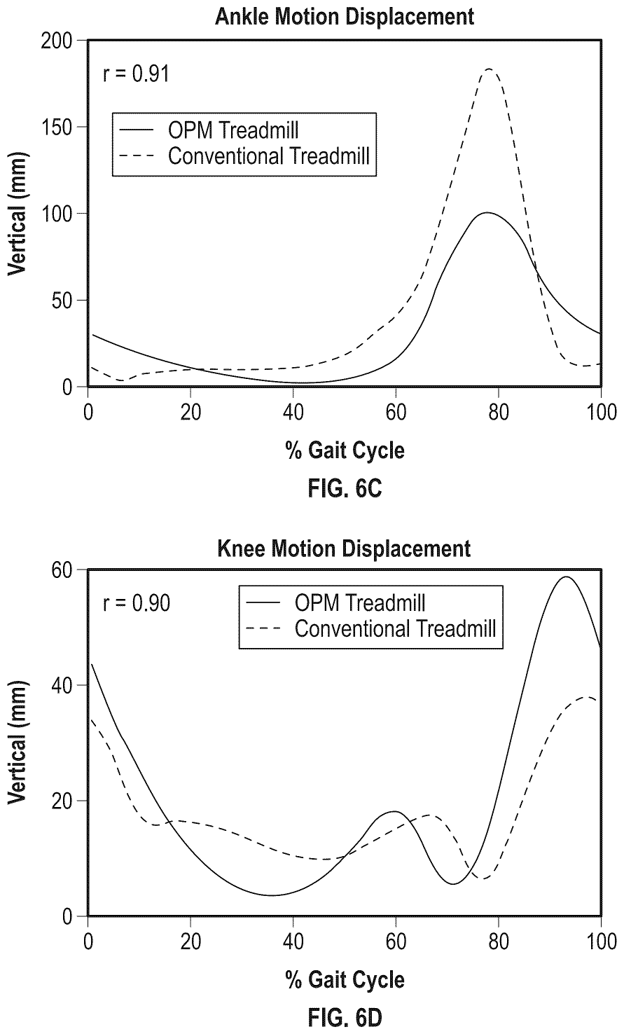

As noted, FIG. 5 shows a toddler walking on the OPM treadmill 12 while a 64-sensor OPM helmet system 32 is used to measure the cortical dynamics during gait. Additionally, triangulations of retroreflective markers are tracked by a high-speed motion capture system that is within the magnetically shielded room and used to calculate the gait biomechanics. Additional results are shown in FIGS. 6A-6D, such as the walking biomechanics for the ankle (6A & 6C) and knee (6B & 6D) while participants walked on a conventional treadmill (red lines) and the OPM treadmill 12 (blue lines). The displayed curve correlations indicate that the biomechanics are remarkably similar (r>0.90).

Preliminary results indicated that the vertical and horizontal displacement of the ankle and knee joint motions were remarkably similar while walking on the respective treadmills (i.e., r>0.90; FIGS. 6A and 6B). As shown in FIGS. 6A and 6B, there were subtle differences in the vertical displacement due to the radius of curvature implemented in the OPM treadmill deck 12 of the present disclosure. Nevertheless, it should be noted that even walking on a conventional treadmill has subtle biomechanical differences when compared with overground walking. Altogether, these results show that walking surface 12 of the present disclosure is an innovative treadmill system that can be used to evaluate the cortical dynamics involved in the emergence of gait in young children and adults alike.

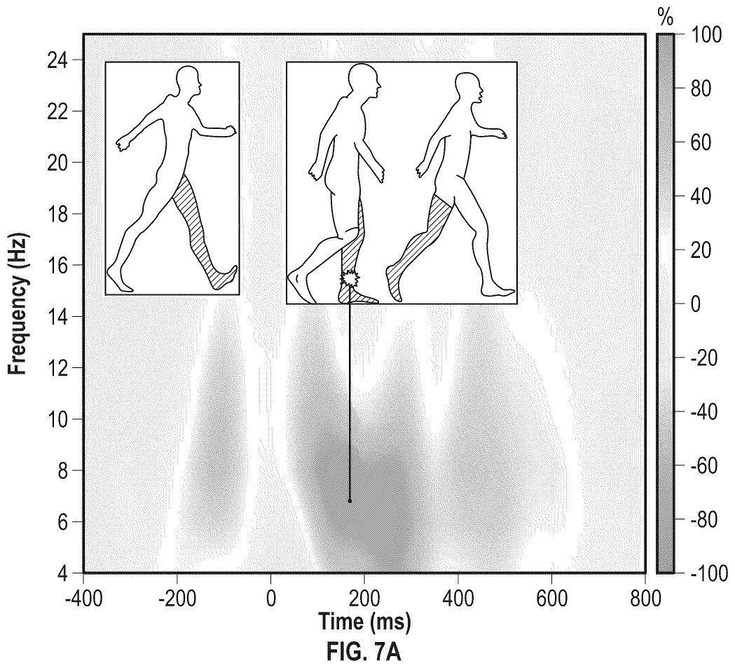

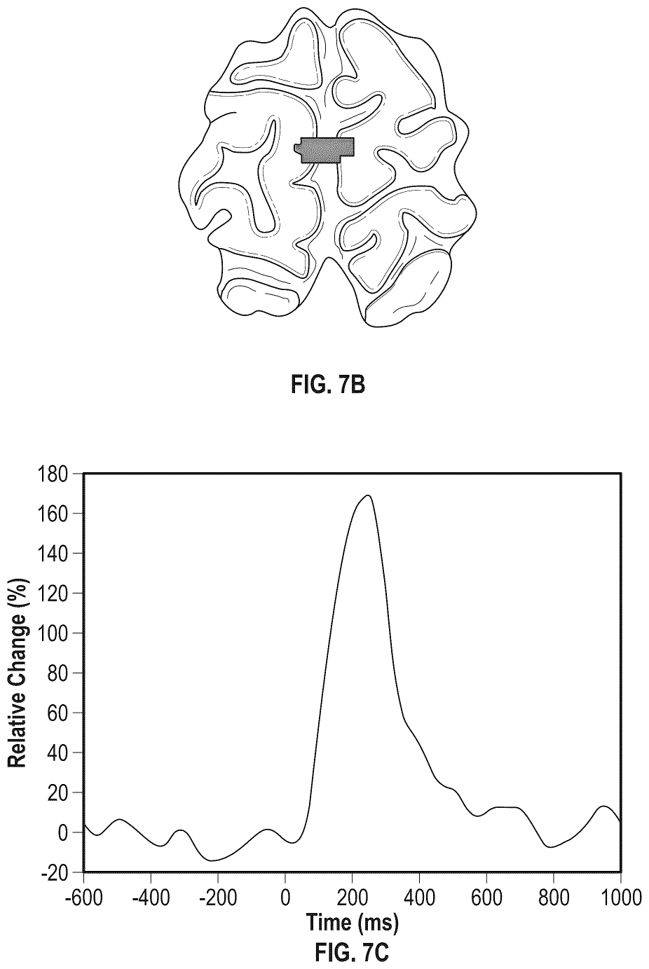

Next, neuroimaging pipes can be used to image the somatosensory cortical dynamics as participants (N=5) walked on the walking surface 12 of the present disclosure. Similar to prior experiments, electrical stimulations of the tibial nerve were applied 200 ms after heel-contact to avoid the artifacts created by the foot colliding with the deck surface. As shown in FIG. 7A, the sensor level time-frequency components displayed a strong theta (4-8 Hz; 50-300 ms) ERS that occurred shortly after the stimulation. The induced activity was flanked by weaker ERS that represented the heel-contacts of the stimulated leg (−200 ms) and the heel-contact of the contralateral leg (500 ms). Hence, highlighting the importance of the experimental design for separating movement related artifacts from the cortical signal. As expected, the source level images confirmed that the theta ERS was centered on the leg region of the sensorimotor cortices (FIG. 7B). The neural time course can be subsequently extracted from the peak voxel in the group averaged beamformed image to further substantiate the robustness of the neuroimaging pipeline and rigor of the OPM treadmill 12. As shown in FIG. 7C, there was a strong increase in the somatosensory cortical activity while walking on the OPM treadmill 12. These preliminary results show that the use of the walking system 10 of the present disclosure in combination with an OPM will provide groundbreaking insights on the changes in the sensorimotor cortical activity in children younger than 7 years.

In addition, while the use of the components for children under 7 years of age has been identified, it should also be noted that the treadmill as disclosed could be used with a participant of any age, including adults. It should be noted that the example provided in FIG. 9 is an adult user, showing that the system can be used for any age. Such information could also be used for diagnostic purposes or monitoring the effectiveness of therapeutics (i.e., physical therapy, and/or drug treatments).

Still additional aspects, embodiments, features, and the like may be included. The position of the walking surface should not substantially move or rotate in comparison to the frame when someone is walking on the treadmill or hanging from the harness. To achieve this, there may or may not be a common base in which the frame and the walking surface are attached to. The frame and walking surface may be attached with mechanical or non-mechanical fasteners to the common base if there is one. If there is not a common base the frame and the walking apparatus may be attached together mechanically or non-mechanically.

The bottom of the treadmill should be either mechanically fastened, non-mechanically fastened to the room in which it is in, or the friction force must be high enough were the treadmill does not move or rotate in comparison to the room in which the OPM scan is being done when a person is walking on the treadmill.

The harness should attach to the frame at a distance where the user is restricted in their ability to walk off of the walking surface but should be attached at a distance loose enough to allow the person to shift their center of mass onto a sloped portion of the walking surface.

Therefore, a walking system for use with an OPM has been shown and/or described. It should be appreciated that variations and/or changes to any of the components or embodiments that are obvious to those skilled in the art are to be considered a part of the present disclosure. In addition, any of the aspects of any of the embodiments disclosed could be combined in ways not explicitly shown and/or described to provide yet additional embodiments that are part of the disclosure. The disclosure is not to be limited to the embodiments disclosed herein.

1. A walking system for use with an optically pumped magnetometer, comprising:
a walking surface comprising a center and one or more upward sloping surfaces extending away from the center;
wherein the walking surface comprises a low-friction material; and
a frame adjacent to the walking surface, the frame including a harness for a user to aid in controlling the location of the user relative to the walking surface.

2. The walking system of claim 1, further comprising shoes for lowering the coefficient of friction while contacting the walking surface.

3. The walking system of claim 2, wherein the coefficient of friction between the shoes and the walking surface is about 0.03-0.40.

4. The walking system of claim 1, wherein the harness is a waistband, chest, or full body harness.

5. The walking system of claim 1, wherein the walking surface is circular-shaped.

6. The walking system of claim 1, wherein the one or more upward sloping surfaces of the walking surface is substantially the same slope around the center.

7. The walking system of claim 1, wherein the walking surface comprises a low-friction polymer.

8. The walking system of claim 1, wherein the system is substantially free of ferromagnetic materials.

9. A method of collecting data using an optically pumped magnetometer while a user moves, comprising:
connecting a user to the optically pumped magnetometer;
having the user walk on a walking apparatus, wherein the walking apparatus comprises a walking surface comprising a center and upward sloping surfaces extending away from the center and the walking surface comprising a low-friction material; and
maintaining the user's position on the walking apparatus with a harness connected to a frame.

10. The method of claim 9, further comprising providing shoes to the user, wherein the coefficient of friction between the shoes and the walking surface is about 0.03-0.40.

11. The method of claim 9, further comprising collecting data via the optically pumped magnetometer as the user walks on the walking surface.

12. The method of claim 9, wherein the step of connecting the user comprises placing a helmet with sensors on the user's head.

13. The method of claim 9, wherein the walking apparatus is circular-shaped.

14. The method of claim 13, wherein the upward sloping surfaces of the walking surface is substantially the same slope around the center.

15. A walking system for use with an optically pumped magnetometer, comprising:
a walking surface comprising a center and an upwardly sloped surface extending from the center, said walking surface comprising a low-friction material;
a frame adjacent the walking surface including one or more support members and a harness connecting a user to the one or more support members; and
walking shoes for use with the walking surface, the walking shoes comprising a material to lower the coefficient of friction between the walking shoes and the walking surface.

16. The walking system of claim 15, wherein the system is substantially free of ferromagnetic materials.

17. The walking system of claim 15, wherein the coefficient of friction between the shoes and the walking surface is about 0.03-0.40.

18. The walking system of claim 15, wherein the upwardly sloped surface of the walking surface comprises a continuous surface circling the center.

19. The walking system of claim 18, wherein the slope of the continuous surface circling the center is substantially the same slope around the center.

20. The walking system of claim 15, wherein the harness is a waistband, chest, or full body harness.
